(19) United States
(12) Patent Application Publication
(10) Pub. No.: US 2006/0167032 A1
(43) Pub. Date: Jul. 27, 2006

(54) Pharmaceutical composition and method for treating disorders of the central nervous system

(76) Inventors: Bradley S Galer, West Chester, PA (US); Thomas G Schlagheck, Naples, FL (US)

Correspondence Address: DILWORTH & BARRESE, LLP, 333 EARLE OVINGTON BLVD., UNIONDALE, NY, 11553, US

(21) Appl. No.: 10/501,678

(22) PCT Filed: Jan. 10, 2003
(86) PCT No.: PCT/US03/00794

§ 371 Date: Jul. 12, 2005

Related U.S. Application Data
(60) Provisional application No. 60/349,773, filed on Jan. 16, 2002.

(51) Int. Cl. A61K 31/485; A61K 31/22; A61K 31/195; A61K 31/13; A61K 31/165

(52) U.S. Cl. 514/282; 514/561; 514/551; 514/663; 514/617

(57) ABSTRACT

Disorders of the ventral nervous system (CNS) are treated by the administration of a GABA analog such as gabapentin or pregablin, an NMDA receptor antagonist such as dextromethorphan or d-methodone and, optionally, another pharmacologically active substance, e.g., one which is effective for the treatment of a CNS disorder.

### BACKGROUND

This application claims the benefit under 35 U.S.C. § 119(e) of earlier filed and copending U.S. Provisional Application No. 60/349,773, filed Jan. 16, 2002, the contents of which are incorporated herein by reference.

### BACKGROUND OF THE INVENTION

1. Technical Field

This invention relates to a pharmaceutical composition for treating disorders of the central nervous system (CNS).

2. Background of Related Art

CNS disorders are types of neurological disorders. CNS disorders can be drug induced; can be attributed to genetic predisposition, infection or trauma; or can be of unknown etiology. CNS disorders comprise neuropsychiatric disorders, neurological diseases and mental illnesses, and include neurodegenerative diseases, behavioral disorders, cognitive disorders and cognitive affective disorders. There are several CNS disorders whose clinical manifestations have been attributed to CNS dysfunction, i.e., disorders resulting from inappropriate levels of neurotransmitter release, inappropriate properties of neurotransmitter receptors, and/or inappropriate interaction between neurotransmitters and neurotransmitter receptors. Several CNS disorders can be attributed to a cholinergic deficiency, a dopaminergic deficiency, an adrenergic deficiency and/or a serotonergic deficiency. CNS disorders of relatively common occurrence include presenile dementia (early onset Alzheimer's disease), senile dementia (dementia of the Alzheimer's type), movement disorders associated with Parkinsonism including Parlinson's disease, Restless Leg Syndrome (RLS), Lewy Body Disease (LBD), supranuclear palsy (SNP), Huntington's chorea, tardive dyskinesia, hyperlinesia, mania, attention deficit disorder, depression, anxiety, obsessive-compulsive disorders, dyslexia, schizophrenia, headache disorders such as migraine and cluster headaches, epilepsy and Tourette's syndrome.

GABA analogs are known in the art and include those disclosed, e.g., in U.S. Pat. Nos. 4,024,175, 4,087,544 and 5,563,175, the contents of each of which are incorporated by reference herein.

N-methyl-D-aspartate (NMDA) receptor antagonists are well known in the art and encompass, for example, dextromethorphan, dextrorphan, memantine, amantidine, d-methadone and their pharmaceutically acceptable salts. NMDA receptor antagonists are known to inhibit the development of tolerance to and/or dependence on addictive drugs, e.g., narcotic analgesics such as morphine, codeine, etc., as disclosed in U.S. Pat. Nos. 5,321,012 and 5,556,838, and to treat chronic pain as disclosed in U.S. Pat. No. 5,502,058, the contents of each of which are incorporated by reference herein.

Nontoxic NMDA receptor antagonists, such as dextromethorphan, are also known to enhance the effects of some drugs, especially opioid analgesics. See, e.g., U.S. Pat. Nos. 5,502,058 and 5,840,731, respectively, the contents of which are incorporated by reference herein. In some cases, the nontoxic NMDA receptor antagonist is administered in combination with a local anesthetic. See U.S. Pat. No. 5,352,683, the contents of which are incorporated by reference herein.

It is an object of the present invention to provide a CNS disorder-treating composition and method in which the CNS disorder-treating activity of a GABA analog is potentiated by a nontoxic NMDA receptor antagonist.

It is another object of the present invention to provide a CNS disorder-treating single unit dosage form containing a GABA analog, at least one nontoxic NMDA receptor antagonist and, optionally, one or more additional pharmacologically active substances, e.g., another drug which is effectual for treatment of a CNS disorder.

### SUMMARY OF THE INVENTION

By way of meeting the foregoing as well as other objects of the invention, a method of treating a CNS disorder is provided which comprises administering to a mammal in need of treatment for a CNS disorder a pharmaceutical composition which comprises (a) at least one GABA analog and (b) at least one nontoxic antagonist, or blocker, for the N-methyl-D-aspartate (NMDA) receptor, the combined amount of (a) and (b) in the composition being a CNS disorder-treating amount and the amount of (b) in the composition being sufficient to potentiate the CNS disorder-treating effectiveness of (a). Optionally, the pharmaceutical composition utilized in the methods of the present invention may include a third component, (c), which is a therapeutically effective amount of at least one other CNS disorder-treating drug or other pharmacologically active substance.

The expression “NMDA receptor antagonist” shall be understood herein to be synonymous with, and to include, the expressions “antagonist for the NMDA receptor” and “blocker for the NMDA receptor”, and shall be understood to include all nontoxic substances that block an NMDA receptor binding site.

The term “nontoxic” as used herein shall be understood in a relative sense and is intended to designate any substance that has been approved by the United States Food and Drug Administration (“FDA”) for administration to humans or, in keeping with established regulatory criteria and practice, is susceptible to approval by the FDA for administration to humans. The term “nontoxic” is also used herein to distinguish the NMDA receptor antagonists that are useful in the practice of the present invention from NMDA receptor antagonists such as MK 801 (the compound 5-methyl-10,11-dihydro-SH-dibenze[a,d]cyclohepten-5,10-imine), CPP (the compound 3-[2-carboxypiperazin-4-yl]propyl-1-phosphonic acid) and PCP (the compound 1-(1-phenylcyclohexyl) piperidine) whose toxicities effectively preclude their therapeutic use.

The expression “CNS disorder-treating” shall be understood herein to be synonomous with, and to include, the expressions “CNS disorder-alleviating”, “CNS disorder-suppressing” and “CNS disorder-inhibiting”, as the CNS disorder-treating method of the invention is applicable to the alleviation of an existing CNS disorder as well as the suppression or inhibition of a CNS disorder in a subject known to manifest such a disorder.

The term “potentiate”, as applied to the pharmaceutical composition and CNS disorder-treating method of the invention, shall mean that the presence of the nontoxic NMDA receptor antagonist in the CNS disorder-treating composition does one of the following: (i) increases CNS disorder-treating effects so that the CNS disorder-treating effects from the composition of the present invention is greater than the sum of the CNS disorder-treating effects attributable to its GABA analog and nontoxic NMDA receptor antagonist components when each of these components is administered alone, (ii) provides the same level of CNS disorder-treating effects using a lower amount of GABA analog compared to the GABA analog alone, (iii) creates a synergistic effect when administered with the GABA analog so that CNS disorder-treating effects are obtained when the CNS disorder-treating composition of the present invention is administered, but would not be obtained if the nontoxic NMDA receptor antagonist and GABA analog were administered alone and to the exclusion of the other; (iv) suppresses or minimizes any adverse effects of the GABA analog.

### DESCRIPTION OF THE PREFERRED EMBODIMENTS

The present invention is useful for treating many CNS disorders, including those mentioned above. Some of the CNS disorders treatable by a pharmaceutical composition in accordance with this invention as classified in the International Classification of Diseases of the World Health Organization are as follows:

Dementia in Alzheimer's Disease

F01 Vascular dementia
F02 Dementia in other diseases classified elsewhere
F05 Delirium, not induced by alcohol and other psychoactive substances
F06 Other mental disorders due to brain damage and dysfunction and to physical disease
F06.0 Organic hallucinosis
F06.2 Organic delusional [schizophrenia-like] disorder
F06.3 Organic mood [affective] disorder
F06.4 Organic anxiety disorder
F06.7 Mild cognitive disorder
F07.1 Postencephalitic syndrome
F07.2 Postconcussional syndrome
F11. Mental and behavioural disorders due to use of opioids
F12. Mental and behavioural disorders due to use of cannabinoids
F13. Mental and behavioural disorders due to use of sedatives or hypnotics
F14. Mental and behavioural disorders due to use of cocaine
F16. Mental and behavioural disorders due to use of hallucinogens
F17. Mental and behavioural disorders due to use of tobacco
   Schizophrenia
   Manic Episode
F30.0 Hypomania
F30.1 Mania without psychotic symptoms
F30.2 Mania with psychotic symptoms
F30.8 Other manic episodes
F30.9 Manic episode, unspecified
F31 Bipolar affective disorder
F31.0 Bipolar affective disorder, current episode hypomanic
F31.1 Bipolar affective disorder, current episode manic without psychotic symptoms
F31.2 Bipolar affective disorder, current episode manic with psychotic symptoms
F31.3 Bipolar affective disorder, current episode mild or moderate depression
F31.4 Bipolar affective disorder, current episode severe depression without psychotic symptoms
F31.5 Bipolar affective disorder, current episode severe depression with psychotic symptoms
F32 Depressive episode
F34 Persistent mood [affective] disorders
F34.0 Cyclothymia
F34.1 Dysthymia
F41 Other anxiety disorders
F41.0 Panic disorder [episodic paroxysmal anxiety]
F41.1 Generalized anxiety disorder
F41.2 Mixed anxiety and depressive disorder
F41.3 Other mixed anxiety disorders
F41.8 Other specified anxiety disorders
F41.9 Anxiety disorder, unspecified
F42 Obsessive-compulsive disorder
F43.1 Post-traumatic stress disorder
F43.2 Adjustment disorders
F51 Nonorganic sleep disorders
F55 Abuse of non-dependence-producing substances
F55.0 Antidepressants
F55.2 Analgesics
F61 Mixed and other personality disorders
F63 Habit and impulse disorders
   Diseases of the Nervous System (G00-G99)
   Seasonal Affective Disorder

Additional CNS disorders that are treatable in accordance with the invention include:

AIDS—Neurological Manifestations

Acquired Epileptiform Aphasia

Amyotrophic Lateral Sclerosis

Anoxia or Hypoxia

Apraxia

Attention Deficit-Hyperactivity Disorder

Autism

Brain Injury

Cerebral Palsy

Chorea

Dementia with Lewy Bodies

Encephalitis and Meningitis

Encephaloceles

Epilepsy

Head Injury

Herpes Zoster

Hypoxia

Immune-Mediated Encephalomyelitis

Kuru

Lennox-Gastaut Syndrome

Leukodystrophy

Lewy Body Dementia

Lissencephaly

Locked-In Syndrome

Lou Gehrig's Disease

Lupus—Neurological Sequelae

Lyme Disease—Neurological Sequelae

Meningitis

Motor Neuron Diseases

Moyamoya Disease

Multiple System Atrophy with Postural Hypotension

Narcolepsy

Neurofibromatosis

Neurological Manifestations of AIDS

Neurological Sequelae Of Lupus

Neurological Sequelae Of Lyme Disease

Niemann-Pick Disease

Parkinson's Disease

Pick's Disease

Post-Polio Syndrome

Postinfectious Encephalomyelitis

Progressive Supranuclear Palsy

Pseudotumor Cerebri

Restless Legs Syndrome

Schilder's Disease

Sydenham Chorea

Syncope

Systemic Lupus Erythematosus

Tardive Dyskinesia

Tremor

Wilson's Disease

In addition to the foregoing CNS disorders, other CNS disorders, or neurological diseases, that may be usefully treated by the pharmaceutical composition of this invention include, but are not limited to, Tourette's syndrome, Asperger's syndrome, and similar genetic and infectious diseases affecting the brain and/or spinal cord.

Useful GABA analogs include those disclosed, e.g., in U.S. Pat. Nos. 4,024,175, 4,087,544 and 5,563,175. A preferred embodiment of the therapeutic composition herein utilizes a GABA analog of Formula I 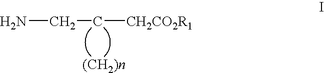
wherein R₁ is hydrogen or lower alkyl and n is an integer of from 4 to 6, and the pharmaceutically acceptable salts thereof.

A preferred GABA analog of Formula I wherein R₁ is hydrogen and n is 5 is the compound 1-(aminomethyl)-cyclohexane acetic acid, known generically as gabapentin. Other preferred GABA analogs of Formula I wherein the cyclic ring is substituted, for example, with alkyl such as methyl or ethyl, include such compounds as (1-aminomethyl-3-methylcyclohexyl)acetic acid, (1-aminomethyl-3-methylcyclopentyl) acetic acid, and (1-aminomethyl-3,4-dimethylcyclopentyl) acetic acid.

Another preferred embodiment of the therapeutic composition herein utilizes a GABA analog of Formula II 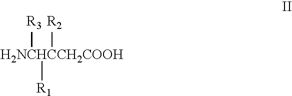
wherein R₁ is a straight or branched alkyl of from 1 to 6 carbon atoms, phenyl, or cycloaklyl of from 3 to 6 carbon atoms, R₂ is hydrogen or methyl and R₃ is hydrogen, methyl, or carboxyl, and the pharmaceutically acceptable salts, diastereomers and enantiomers thereof.

Preferred GABA analogs of Formula II are those wherein R₂ and R₃ are both hydrogen and R₁ is —(CH₂)₀₋₂-iC₄H₉ as an (R), (S), or (R,S) isomer. A preferred compound of this type is 3-aminomethyl-5-methyl-hexanoic acid, and especially (S)-3-(aminomethyl)-5-methylhexanoic acid, known generically as pregabalin, Pregabalin is also known as “CI-1008” and “S-(+)-3-IBG.” Another preferred compound of Formula II is 3-(1-aminoethyl)-5-methylheptanoic acid.

GABA analogs, including those described above, are readily available, either commercially or by synthetic methodology well-known to those skilled in the art of organic chemistry.

Among the nontoxic substances that block the NMDA receptor and as such are useful for potentiating the CNS disorder-treating activity of the GABA analog in accordance with this invention are dextromethorphan ((+)-3-hydroxy-N-methylmorphinan), its metabolite dextrorphan ((+)-3-hydroxy-N-methylmorphinan), amantadine (1-amino adamantine), memantine (3,5 dimethylaminoadamantone), d-methadone (d-form of 6-dimethylamino-4,4-diphenyl-3-heptanone hydrochloride), their mixtures and their pharmaceutically acceptable salts.

Of the foregoing NMDA-receptor antagonists, dextromethorphan is preferred due to its wide use in over-the-counter medications where it functions as a cough suppressant.

For purposes of this disclosure, “extended release” includes “controlled release” and “sustained release” and pertains to the release of pharmaceutical agents at a defined level over an extended period of time.

The expression “dosage form” is understood to include “unit dosage form”. The expression “unit dosage form” means a physically discrete unit which contains specified amounts of a GABA analog in combination with the nontoxic NMDA receptor antagonist, and any other pharmacologically active substance or pharmaceutical excipient, which amounts are selected so that a fixed number, e.g. one, of the units is suitable to achieve a desired therapeutic effect.

In the pharmaceutical composition of this invention, the combined amount of GABA analog and nontoxic NMDA receptor antagonist must be a CNS disorder-treating amount, the amount of NMDA receptor antagonist in the composition being sufficient to potentiate the CNS disorder-treating activity of the GABA analog component of the composition. The GABA analog can be present in the pharmaceutical composition in an amount which, if administered by itself, would constitute a CNS disorder-treating amount or it can be present in the composition in less than this amount provided that the amount of nontoxic NMDA receptor antagonist present therein is sufficient to provide an effective CNS disorder-treating dose.

As noted above, the nontoxic NMDA receptor antagonist must be present in the pharmaceutical composition in an amount sufficient to potentiate the CNS disorder-treating activity of the GABA analog. It would be recognized by one skilled in the art that this amount will relate to the amount of the GABA analog present and its CNS disorder-treating capacity, the nature of the nontoxic NMDA receptor antagonist and its ability to enhance the CNS disorder-treating effect, as well as the particular formulation containing the active substances. As those skilled in the art will recognize, many factors that modify the action of the active substances herein, such as the state and circumstances of the host being treated, will be taken into account by the treating physician and include, for example, the age, body weight, sex, diet and condition of the subject, including metabolic status, the time of administration, the rate and route of administration, and so forth. Optimal dosages for a given set of conditions can be ascertained by those skilled in the art using conventional dosage determination tests.

A useful intravenous combined dosage form can, e.g., contain from about 5 to about 50 mg of the selected GABA analog and a useful oral dosage form can contain form about 10 to about 800 mg of the GABA analog. Given these wide variations in dosage levels of the GABA analog component, there can similarly be a wide variation in the dosage level of the nontoxic NMDA receptor antagonist. For the preferred nontoxic NMDA receptor antagonist, dextromethorphan in the form of its hydrobromide salt, dosages can generally range from about 10 mg to about 750 mg per 70 kg body weight, preferably from about 30 mg to about 500 mg per 70 kg body weight.

In addition to the GABA analog and at least one nontoxic NMDA receptor antagonist, the pharmaceutical composition herein can optionally contain at least one other pharmacologically active substance which is useful for the treatment of CNS disorders, including any of the specific CNS disorders mentioned above. Illustrative, but not exclusive, of such other pharmaceutically active substances and the specific CNS disorders for which they are indicated to be useful are:
Antidepressants, including trazodone,
   Tricyclics, including amitriptyline (Elevil™), desipramine (Norpramin™), doxepin (Sinequan™ or Adapin™), imipramine (Tofranil™), nortriptyline (Aventyl™ or Pamelor™), clomiprimine (Anafranil™)
   selective serotonin re-uptake inhibitors (SSRIs), including citalopram (Celexa™), fluoxetine (Prozac™), fluvoxamine (Luvox™), paroxetine (Paxil™), sertraline (Zoloft™), temazepam (Restoril™)
   Norepinephrine Serotonin Reuptake Inhibitors (“NSRIs”), including venlafaxine (Effexor™), mirtazapine (Remeron™), nefazodone (Serzone™), milnacipran, and duloxetine (Cymbalta®)
   buspirone (BuSpar®)
   bupropion hydrochloride (Wellbutrin®)
   Dopaminergic, including levodopa and levodopa in combination with carbidopa
Memory-enhancing or Memory-stabilizing
   acetylcholinesterase (AChE) inhibitors, including tacrine and donezepil; selegiline
Antipsychotic and/or Antischizophrenic, including chlorpromazine (Thorazine™), haloperidol (Haldol™), pimozide, fluphenazine
Anti-addiction drugs
   Opioid antagonists
   Dopaminergic
   Nicotinergic, including nicotine and nicotinic compounds
Riluzole
methylphenidate (Ritalin)
Parkinsonian drugs
   Methyldopa
   Anticholinergic
   Dopaminergic, including bromocriptine,
Adrenergic agonists, including clonidine
Anti-anxiety drugs, including benzodiazepines and clonazepam

These and other drugs for treating CNS disorders can be included in the pharmaceutical composition of this invention at known and conventional dosage levels. It will also be apparent to one skilled in the art that many of the drugs noted above can be classified in more than one category for more than one use. Thus, for example, clonidine, which was originally marketed for the treatment of hypertension, has found uses in treating opiate withdrawal, anxiety, and attention deficit disorder.

The pharmaceutical composition of this invention will ordinarily be formulated with one or more pharmaceutically acceptable ingredients in accordance with known and established practice. Thus, the pharmaceutical composition can be formulated as a liquid, powder, elixir, injectable solution, etc. Formulations for oral use can be provided as tablets or hard capsules wherein the pharmacologically active ingredients are mixed with an inert solid diluent such as calcium carbonate, calcium phosphate or kaolin, or as soft gelatin capsules wherein the active ingredients are mixed with an oleaginous medium, e.g., liquid paraffin or olive oil. Formulations include those of immediate-release preparations and those providing modified-release or extended release characteristics, such as those that provide dosing every 6 hours, every 8 hours, every 12 hours, every 24 hours, up to those that provide dosing intervals up to a monthly basis.

While it is within the scope of the invention to concurrently administer separate dosage forms of GABA analog and the nontoxic NMDA receptor antagonist to treat a CNS disorder, as a matter of convenience these drugs are preferably coadministered as a single, or combined, dosage form. All modes of administration are contemplated, e.g., orally, rectally, parenterally, intrathecally, intranasally, transdermally, and topically. The term parenteral as used herein includes subcutaneous, intravenous, intramuscular and intrasternal injections or infusion techniques. In addition to the treatment of warm-blooded animals such as mice, rats, horses, cattle, sheep, dogs, cats, etc., the compounds of the invention are effective in the treatment of humans.

As the pharmaceutical compositions of the present invention contain at least two components, the pharmaceutical compositions may provide for the immediate release of the GABA analog and the NMDA receptor antagonist, the extended release of the two components by inclusion in the same or different sustained release carrier(s), or, in some cases, the immediate release of one component and the extended release of the other component. Similarly, where at least one other drug for treating a CNS disorder or a therapeutically effective amount of at least one other pharmacologically active substance is included in the pharmaceutical composition in addition to the GABA analog and the at least one nontoxic NMDA receptor antagonist, the pharmaceutical composition may provide for the immediate release of the components, the extended release of the components by inclusion in the same or different sustained release carrier(s), or the immediate release of some component(s) and the extended release of the other component(s).

Sustained release of the pharmaceutical composition may be accomplished in accordance with formulations/methods of manufacture known to those skilled in the art of pharmaceutical formulation, e.g., via the incorporation of the pharmaceutical composition in an extended release carrier; or via a controlled release coating of a carrier containing the pharmaceutical composition.

In one embodiment, the pharmaceutical composition comprises a GABA analog in an extended release form in combination with at least one nontoxic NMDA receptor antagonist in an unmodified state capable of immediate release. In another embodiment, an extended release carrier containing the GABA analog is combined with an immediate release carrier containing the nontoxic NMDA receptor antagonist. The nontoxic NMDA receptor antagonist may also be applied to the exterior surface of the extended release carrier and is thus available for immediate release. Alternatively, the GABA analog may be contained in a normal release carrier having a coating that controls the release of the drug. In such a case, the coating may contain the nontoxic NMDA receptor antagonist, which is available for immediate release. Where at least one other drug for treating a CNS disorder or one other therapeutically effective amount of at least one other pharmacologically active substance is included in the analgesic composition in addition to the GABA analog and at least one nontoxic NMDA receptor antagonist, the other drug may be included in either the extended release carrier, the immediate release carrier, or both, depending upon the pharmacologically active substance and its desired effect(s).

Suitable base materials for controlled release carriers include combinations of higher aliphatic alcohols and acrylic resins. Base compositions prepared from such higher aliphatic alcohols and acrylic resins provide sustained release of therapeutically active ingredients over a period of time from five hours and for as much as 24 hours after administration, generally oral administration, in humans or animals.

These bases can be prepared from any pharmaceutically acceptable higher aliphatic alcohol, the most preferred being fatty alcohols of 10-18 carbon atoms, particularly stearyl alcohol, cetyl alcohol, cetostearyl alcohol, lauryl alcohol, myristyl alcohol and mixtures thereof.

Any acrylic polymer which is pharmaceutically acceptable can be used for the purposes of the present invention. The acrylic polymers may be cationic, anionic or non-ionic polymers and may be acrylates or methacrylates, formed of methacrylic acid or methacrylic acid esters. These polymers can be synthesized, as indicated above, to be cationic, anionic or non-ionic, which then renders the polymers pH dependent and consequently soluble in, or resistant to, solutions over a wide range in pH.

In addition, suitable materials for inclusion in a controlled release carrier include:

(a) Hydrophilic polymers, such as gums, cellulose ethers, acrylic resins and protein derived materials. Of these polymers, the cellulose ethers, especially hydroxyalkylcelluloses and carboxyalkylcelluloses, are preferred. The analgesic composition may contain between 1% and 80% (by weight) of at least one hydrophilic or hydrophobic polymer.

(b) Digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, and waxes. Hydrocarbons having a melting point of between 25° and 90° C. are preferred. Of these long chain hydrocarbon materials, fatty (aliphatic) alcohols are preferred. The oral dosage form may contain up to 60% (by weight) of at least one digestible, long chain hydrocarbon.

(c) Polyalkylene glycols. The oral dosage form may contain up to 60% (by weight) of at least one polyalkylene glycol.

One particularly suitable carrier comprises at least one water soluble hydroxyalkyl cellulose, at least one C₁₂-C₃₆, preferably C₁₄-C₂₂, aliphatic alcohol and, optionally, at least one polyalkylene glycol.

The at least one hydroxyalkyl cellulose is preferably a hydroxy (C₁ to C₆) alkyl cellulose, such as hydroxypropylcellulose, hydroxypropylmethylcellulose and, especially, hydroxyethyl cellulose. The amount of the at least one hydroxyalkyl cellulose in the present pharmaceutical composition will be determined, inter alia, by the precise rate of drug release required. Preferably however, the oral dosage form contains between 1% and 45%, especially between 5% and 25% (by weight) of the at least one hydroxyalkyl cellulose.

While the at least one aliphatic alcohol may be, for example, lauryl alcohol, myristyl alcohol or stearyl alcohol, in particularly preferred embodiments the at least one aliphatic alcohol is cetyl alcohol or cetostearyl alcohol. The amount of the at least one aliphatic alcohol in the present dosage form will be determined, as above, by the precise rate of drug release required. It will also depend on whether at least one polyalkylene glycol is present in or absent from the dosage form. In the absence of at least one polyalkylene glycol, the dosage form preferably contains between 20% and 50% (by weight) of the at least one aliphatic alcohol. When at least one polyalkylene glycol is present in the dosage form, then the combined weight of the at least one aliphatic alcohol and the at least one polyalkylene glycol preferably constitutes between 20% and 50% (by weight) of the total dosage.

In the present preferred dosage form, the ratio of, e.g., the at least one hydroxyalkyl cellulose or acrylic resin to the at least one aliphatic alcohol/polyalkylene glycol determines, to a considerable extent, the release rate of the drug from the formulation. A ratio of the at least one hydroxyalkyl cellulose to the at least one aliphatic alcohol/polyalkylene glycol of between 1:2 and 1:4 is preferred, with a ratio of between 1:3 and 1:4 being particularly preferred.

The at least one polyalkylene glycol may be, for example, polypropylene glycol or polyethylene glycol, which is preferred. The number average molecular weight of the at least one polyalkylene glycol is preferred between 1000 and 15000, more preferably between 1500 and 12000.

Another suitable controlled release carrier comprises an alkylcellulose (especially ethyl cellulose), a C₁₂ to C₃₆ aliphatic alcohol and, optionally, a polyalkylene glycol.

In addition to the above ingredients, a controlled release carrier may also contain suitable quantities of other materials, e.g., diluents, lubricants, binders, granulating aids, colorants, flavorants and glidants that are conventional in the pharmaceutical art.

As an alternative to a controlled release carrier, the pharmaceutical composition may be in a normal release carrier having a coating that controls the release of the composition. In particularly preferred embodiments of this aspect of the invention, the present dosage form comprises film coated spheroids containing the pharmaceutical composition and a non-water soluble spheronising agent. The term spheroid is known in the pharmaceutical art and means a spherical granule having a diameter of between 0.5 mm and 2.5 mm especially between 0.5 mm and 2.0 mm.

The spheronising agent may be any pharmaceutically acceptable material that, together with the active ingredient, can be spheronised to form spheroids. Microcrystalline cellulose is preferred. According to a preferred aspect of the present invention, the film coated spheroids contain between 70% and 99% (by weight), especially between 80% and 95% (by weight), of the spheronising agent, especially microcrystalline cellulose.

In addition to the active ingredient(s) and spheronising agent, the spheroids may also contain a binder. Suitable binders, such as low viscosity, water soluble polymers, are well known to those skilled in the pharmaceutical art. However, water soluble hydroxy lower alkyl celluloses, such as hydroxy propyl cellulose, are preferred. Additionally (or alternatively) the spheroids may contain a water insoluble polymer, especially an acrylic polymer, an acrylic copolymer, such as a methacrylic acid-ethyl acrylate copolymer, or ethyl cellulose.

The spheroids are preferably film coated with a material that permits release of the pharmaceutical composition at a controlled rate in an aqueous medium. The film coat is chosen so as to achieve, in combination with the other ingredients, a desirable in vitro release rate, preferably between about 12.5% and about 42.5% (by weight) release after 1 hour.

The film coat will generally include a water insoluble material such as: (a) a wax, either alone or in admixture with a fatty alcohol; (b) shellac or zein; (c) a water insoluble cellulose, especially ethyl cellulose; (d) a polymethacrylate.

Preferably, the film coat comprises a mixture of the water insoluble material and a water soluble material. The ratio of water insoluble to water soluble material is determined by, among other factors, the release rate required and the solubility characteristics of the materials selected.

The water soluble material may be, for example, polyvinylpyrrolidone or, more preferably, a water soluble cellulose, especially hydroxypropylmethyl cellulose.

Suitable combinations of water insoluble and water soluble materials for the film coat include shellac and polyvinylpyrrolidone or, more preferably, ethyl cellulose and hydroxypropylmethyl cellulose. The nontoxic NMDA receptor antagonist may be applied to the exterior surface of, or included within, the film coat to provide for the immediate release of the nontoxic NMDA receptor antagonist while at the same time providing for the extended release of the GABA analog from the spheroid.

In another embodiment, in order to obtain a sustained release of the pharmaceutical composition sufficient to provide a CNS disorder-treating effect for an extended duration, the substrate comprising the pharmaceutical composition may be coated with a sufficient amount of hydrophobic material to obtain a weight gain level from about 2 to about 30 percent, although the overcoat may be greater depending upon the physical properties of the particular pharmaceutical composition and the desired release rate, among other things. In such a case, the GABA analog may be contained in the substrate and the nontoxic NMDA receptor antagonist may be applied to the exterior surface of, or included within, the hydrophobic coating to provide for the immediate release of the nontoxic NMDA receptor antagonist while at the same time providing for the extended release of the GABA analog.

The solvent which is used for the hydrophobic material may be any pharmaceutically acceptable solvent, including water, methanol, ethanol, methylene chloride and mixtures thereof. It is preferable however, that the coatings be based upon aqueous dispersions of the hydrophobic material.

In certain preferred embodiments of the present invention, the hydrophobic polymer comprising the sustained-release coating is a pharmaceutically acceptable acrylic polymer, including but not limited to acrylic acid and methacrylic acid copolymers, methacrylic acid copolymers, methyl methacrylate copolymers, ethoxyethyl methacrylates, cynaoethyl methacrylate, aminoalkyl methacrylate copolymer, polyacrylic acid, polymethacrylic acid, methacrylic acid alkylamide copolymer, poly(methyl methacrylate), methyl methacrylate, polymethacrylate, poly(methyl methacrylate) copolymer, polyacrylamide, poly(methacrylic acid anhydride), and glycidyl methacrylate copolymers.

In other preferred embodiments, the hydrophobic polymer which may be used for coating the substrates of the present invention is a hydrophobic cellulosic material such as ethylcellulose. Those skilled in the art will appreciate that other cellulosic polymers, including other alkyl cellulosic polymers, may be substituted for part or all of the ethylcellulose included in the hydrophobic polymer coatings of the present invention.

In embodiments of the present invention where the coating comprises an aqueous dispersion of a hydrophobic polymer, the inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is especially preferred.

Examples of suitable plasticizers for the acrylic polymers of the present invention include citric acid esters such as triethyl citrate NF XVI, tributyl citrate, dibutyl phthalate, and possibly 1,2-propylene glycol, polyethylene glycols, propylene glycol, diethyl phthalate, and triacetin, although it is possible that other water-insoluble plasticizers (such as acetylated monoglycerides, phthalate esters, castor oil, etc.) may be used. Triethyl citrate is especially preferred.

Sustained-release spheroids or beads, coated with a therapeutically active agent, i.e., pharmaceutical composition, are prepared, e.g., by dissolving the pharmaceutical composition in water and then spraying the solution onto a substrate using a Wurster insert. Optionally, additional ingredients are also added prior to coating the beads in order to assist the pharmaceutical composition binding to the substrates, and/or to color the solution, etc. For example, a product which includes hydroxypropyl methylcellulose, with or without colorant, may be added to the solution and the solution mixed (e.g., for about 1 hour) prior to application of the same onto the beads. The resultant coated substrate, in this example beads, may then be optionally overcoated with a barrier agent, to separate the pharmaceutical composition from the hydrophobic sustained-release coating. An example of a suitable barrier agent is one which comprises hydroxypropyl methylcellulose. However, any film-former known in the art may be used. It is preferred that the barrier agent does not affect the dissolution rate of the final product.

The plasticized aqueous dispersion of hydrophobic polymer may be applied onto the substrate comprising the pharmaceutical composition by spraying using any suitable spray equipment known in the art. In a preferred method, a Wurster fluidized-bed system is used in which an air jet, injected from underneath, fluidizes the core material and effects drying while the acrylic polymer coating is sprayed thereon. A sufficient amount of the aqueous dispersion of hydrophobic polymer to obtain a predetermined sustained-release of said pharmaceutical composition when said coated substrate is exposed to aqueous solutions, e.g. gastric fluid, is preferably applied, taking into account the physical characteristics of the pharmaceutical composition, the manner of incorporation of the plasticizer, etc. After coating with the hydrophobic polymer, a further overcoat of a film-former is optionally applied to the beads. This overcoat is provided, if at all, in order to substantially reduce agglomeration of the beads.

Next, the coated beads are cured in order to obtain a stabilized release rate of the pharmaceutical composition.

The sustained-release profile of the formulations of the invention can be altered, for example, by varying the thickness of the hydrophobic coating, changing the particular hydrophobic material used, or altering the relative amounts of, e.g., different acrylic resin lacquers, altering the manner in which the plasticizer is added (e.g., when the sustained-release coating is derived from an aqueous dispersion of hydrophobic polymer), by varying the amount of plasticizer relative to hydrophobic polymer, by the inclusion of additional ingredients or excipients, by altering the method of manufacture, etc. As noted above, the nontoxic NMDA receptor antagonist may be applied to the exterior of, or contained within, any coating of a carrier containing a GABA analog to provide for the immediate release of the nontoxic NMDA receptor antagonist while at the same time providing for the extended release of the GABA analog.

The coating solutions of the present invention may contain, in addition to the film-former, plasticizer, and solvent system (i.e., water), a colorant to provide elegance and product distinction. Color may be added to the solution of the pharmaceutical composition instead, or in addition to the aqueous dispersion of hydrophobic polymer.

In another embodiment, the pharmaceutical composition of the present invention is in an aqueous suspension. Aqueous suspensions can include pharmaceutically acceptable excipients such as suspending agents, e.g., sodium carboxymethyl cellulose, methylcellulose, hydroxypropylmethylcellulose, sodium alginate, polyvinylpyrrolidone, gum tragacanth and gum acacia; dispersing or wetting agents such as naturally occurring phosphatides, e.g., lecithin, or condensation products of an alkylene oxide with fatty acids, e.g., polyoxyethylene stearate, or condensation products of ethylene oxide with long chain aliphatic alcohols, e.g., heptadecaethylene-oxycetanol, or condensation products of ethylene oxide with partial esters derived from fatty acids and a hexitol, e.g., polyoxyethylene sorbitol monooleate or condensation products of ethylene oxide with partial esters derived from fatty acids and hexitol anhydrides, e.g., polyoxyethylene sorbitan monooleate. The aqueous suspensions can also contain one or more preservatives, e.g., ethyl- or n-propyl-p-hydroxy benzoate, one or more coloring agents, one or more flavoring agents and one or more sweetening agents, such as sucrose, saccharin or sodium or calcium cyclamate.

The pharmaceutical composition herein can be formulated as a solid, liquid, powder, elixir, injectable solution, etc. When formulated for oral delivery, the combination of drugs herein may be in the form of tablets, liquids, troches, lozenges, quick dissolve tablets, aqueous or oily suspensions, multiparticulate formulations including dispersible powders, granules, carrier spheroids or coated inert beads, emulsions, hard or soft capsules, syrups or elixirs, microparticles (e.g., microcapsules, microspheres and the like), buccal tablets, etc. The pharmaceutical preparations can be sterilized and if desired mixed with auxiliary agents, e.g., lubricants, preservatives, stabilizers, emulsifiers, salts for influencing osmotic pressure buffers, coloring, flavoring and/or aromatic substances and the like. They can also be combined where desired with other active agents, e.g., other analgesic agents. For oral administration, particularly suitable are tablets, dragees, liquids, drops, suppositories, capsules, caplets and gelcaps. The compositions intended for oral use may be prepared according to any method known in the art. When prepared as tablets, the tablets may be uncoated or they may be coated by known techniques for elegance or to further delay release of the active ingredients. Formulations for oral use may also be presented as hard gelatin capsules wherein the active ingredient is mixed with an inert diluent.

The following examples are illustrative of pharmaceutical compositions for the treatment of CNS disorders in accordance with the invention:

| | | Drug Components | | |
|---|---|---|---|---|
| | Dosage | GABA Analog, | Nontoxic NMDA | Other Drug |
| Example | Form | mg | Receptor Antagonist, mg | Component, mg |
| 1 | oral | gabapentin, | dextromethorphan HBr, | — |
| | | 50-800 | 10-750 | |
| 2 | oral | gabapentin, | d-methadone HCl, | — |
| | | 50-800 | 50-800 | |
| 3 | oral | pregabalin, | dextromethorphan HBr, | — |
| | | 10-600 | 10-500 | |
| 4 | oral | pregabalin, | d-methadone HCl, | — |
| | | 10-600 | 10-800 | |
| 5 | oral | gabapentin, | dextromethorphan HBr, | nicotine or nicotinic |
| | | 50-800 | 10-500 | compound, 5-200 |
| 6 | oral | gabapentin, | dextromethorphan HBr, | tacrine HCl, |
| | | 50-800 | 10-500 | 5-50 |
| 7 | oral | gabapentin, | dextromethorphan HBr, | donezepil HCl, |
| | | 50-800 | 10-500 | 5-50 |
| 8 | oral | gabapentin, | dextromethorphan HBr, | carbidopa, 10-100, |
| | | 50-800 | 10-500 | and levodopa, 25-250 |
| 9 | oral | pregabalin, | dextromethorphan HBr, | — |
| | | 10-600 | 10-500 | |
| 10 | oral | pregabalin, | d-methadone HCl, | — |
| | | 10-600 | 10-500 | |
| 11 | injectable | gabapentin, | dextromethorphan HBr, | — |
| | | 10-600 | 10-500 | |
| 12 | injectable | pregabalin, | dextromethorphan HBr, | — |
| | | 10-600 | 10-500 | |

It will be understood that various modifications may be made to the embodiments disclosed herein. Therefore, the above description should not be construed as limiting, but merely as exemplifications of preferred embodiments. For example, NMDA receptor antagonists other than dextromethorphan can be utilized in the CNS disorder-treating pharmaceutical composition described herein. Those skilled in the art will envision other modifications within the scope and spirit of the claims appended hereto.

1. A pharmaceutical composition comprising:
(a) at least one GABA analog and
(b) at least one nontoxic antagonist for the NMDA receptor,
the combined amount of (a) and (b) in the composition being a CNS disorder-treating amount and the amount of (b) in the composition being sufficient to potentiate the CNS disorder-treating effectiveness of (a).

2. The composition of claim 1 wherein the GABA analog possesses the structure 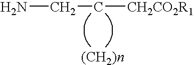
wherein R₁ is hydrogen or lower alkyl and n is an integer of from 4 to 6, and the pharmaceutically acceptable salts thereof.

3. The composition of claim 1 wherein the GABA analog is gabapentin.

4. The composition of claim 1 wherein the GABA analog possesses the structure 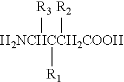
wherein R₁ is a straight or branched alkyl of from 1 to 6 carbon atoms, phenyl, or cycloalkyl of from 3 to 6 atoms, R₂ is hydrogen or methyl and R₃ is hydrogen, methyl, or carboxyl, and the pharmaceutically acceptable salts, diasteromers and enantiomers thereof.

5. The composition of claim 1 wherein the GABA analog is pregabalin.

6. The composition of claim 1 wherein the nontoxic NMDA receptor antagonist is at least one member selected from the group consisting of dextromethorphan, dextrorphan, amantadine, memantine, d-methadone and pharmaceutically acceptable salts thereof.

7-10. (canceled)

11. The composition of claim 1 wherein (a) and (b) of the pharmaceutical composition is present in a combined sustained release carrier.

12. The composition of claim 1 wherein (a) and (b) of the pharmaceutical composition are present in separate sustained release carriers.

13. The composition of claim 1 wherein the pharmaceutical composition contains a therapeutically effective amount of at least one other pharmacologically active substance (c).

14. The composition of claim 1 wherein the pharmaceutical composition contains a therapeutically effective amount of at least one other pharmacologically active substance (c) which is a drug for treating a CNS disorder.

15. The composition of claim 1 wherein the pharmaceutical composition contains a therapeutically effective amount of at least one other pharmaceutically active substance (c) which is a drug or drug combination for the treatment of a CNS disorder selected from the group consisting of nicotine, nicotinic compounds, tacrine, donezepil, carbidopa in combination with levodopa, selegiline, bromocriptine, haloperidol, clonidine, pimozide, fluphenazine, benzodiazepines, clonazepam, clorpromazine, fluoxetine, clomipramine, amitriptyline, nortriptyline, imipramine, buspirone, bupropion hydrochloride, venlafaxine, milnacipran, duloxetine, mirtazapine, nefazodone, paroxetine, sertraline, riluzole, trazodone, doxepin and methylphenidate.

16. The composition of claim 1 wherein the CNS disorder is classified in the International Classification of Diseases of the World Health Organization.

17. The composition of claim 1 wherein the CNS disorder is presenile dementia, senile dementia, movement disorder, hyperkinesias, mania, attention deficit disorder, depression, anxiety, obsessive-compulsive disorder, dyslexia, schizophrenia, headache disorder, epilepsy, Tourette's syndrome or Asperger's syndrome.

18-31. (canceled)

32. A pharmaceutical composition comprising: (a) at least one GABA analog in an extended release form in combination with (b) at least one nontoxic antagonist for the NMDA receptor in an immediate release form, the combined amount of (a) and (b) in the composition being a CNS disorder-treating amount and the amount of (b) in the composition being sufficient to potentiate the CNS disorder-treating effectiveness of (a).

33. The composition of claim 32 wherein the GABA analog possesses the structure 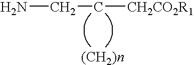
wherein R₁ is hydrogen or lower alkyl and n is an integer of from 4 to 6, and the pharmaceutically acceptable salts thereof.

34. The composition of claim 32 wherein the GABA analog is gabapentin.

35. The composition of claim 32 wherein the GABA analog possesses the structure 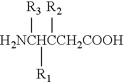
wherein R₁ is a straight or branched alkyl of from 1 to 6 carbon atoms, phenyl, or cycloaklyl of from 3 to 6 carbon atoms, R₂ is hydrogen or methyl and R₃ is hydrogen, methyl, or carboxyl, and the pharmaceutically acceptable salts, diastereomers and enantiomers thereof.

36. The composition of claim 32 wherein the GABA analog is pregabalin.

37. The composition of claim 32 wherein the nontoxic NMDA receptor antagonist is at least one member selected from the group consisting of dextromethorphan, dextrorphan, amantadine, memantine, d-methadone and pharmaceutically acceptable salts thereof.

38-41. (canceled)

42. The composition of claim 32 wherein the at least one nontoxic NMDA receptor antagonist is present in an immediate release carrier.

43. The composition of claim 32 wherein the extended release form is an extended release carrier comprising abase material selected from the group consisting of a hydrophilic polymer, a hydrophobic polymer, a long chain hydrocarbon, a polyalkylene glycol, higher aliphatic alcohols, acrylic resins, and mixtures thereof.

44. The composition of claim 43 wherein the at least one nontoxic NMDA receptor antagonist is applied to the extended release carrier's exterior surface.

45. The composition of claim 32 wherein the extended release form comprises a base material having a coating that controls the release of the GABA analog.

46. The composition of claim 45 wherein the coating includes the at least one nontoxic NMDA receptor antagonist.

47. The composition of claim 32 wherein the pharmaceutical composition contains a therapeutically effective amount of (c) at least one other pharmacologically active substance.

48. The composition of claim 47 wherein the pharmacologically active substance (c) is included in the extended release form.

49. The composition of claim 47 wherein the pharmacologically active substance (c) is included in the immediate release form.

50. The composition of claim 47 wherein the pharmacologically active substance (c) is included in both the extended release form and the immediate release form.

51. The composition of claim 32 wherein the pharmaceutical composition contains a therapeutically effective amount of at least one other pharmacologically active substance (c) which is a drug for treating a CNS disorder.

52. The composition of claim 32 wherein the pharmaceutical composition contains a therapeutically effective amount of at least one other pharmaceutically active substance (c) which is a drug or drug combination for the treatment of a CNS disorder selected from the group consisting of nicotine, nicotinic compounds, tacrine, donezepil, carbidopa in combination with levodopa, selegiline, bromocriptine, haloperidol, clonidine, pimozide, fluphenazine, benzodiazepines, clonazepam, clorpromazine, fluoxetine, clomipramine, amitriptyline, nortriptyline, imipramine, buspirone, bupropion hydrochloride, venlafaxine, milnacipran, duloxetine, mirtazapine, nefazodone, paroxetine, sertraline, riluzole, trazodone, doxepin and methylphenidate.

53. The composition of claim 32 wherein the CNS disorder is classified in the International Classification of Diseases of the World Health Organization.

54. The composition of claim 32 wherein the CNS disorder is presenile dementia, senile dementia, movement disorder, hyperkinesias, mania, attention deficit disorder, depression, anxiety, obsessive-compulsive disorder, dyslexia, schizophrenia, headache disorder, epilepsy, Tourette's syndrome or Asperger's syndrome.

55. A method of treating a CNS disorder which comprises administering to a mammal in need of treatment for a CNS disorder A CNS disorder treating amount of pharmaceutical composition comprising:
(a) at least one GABA analog and
(b) at least one nontoxic antagonist for the NMDA receptor,
the combined amount of (a) and (b) in the composition being a CNS disorder-treating amount and the amount of (b) in the composition being sufficient to potentiate the CNS disorder-treating effectiveness of (a).

56. A method of treating a CNS disorder which comprises administering to a mammal in need of treatment for a CNS disorder A CNS disorder treating amount of pharmaceutical composition comprising: (a) at least one GABA analog in an extended release form in combination with (b) at least one nontoxic antagonist for the NMDA receptor in an immediate release form, the combined amount of (a) and (b) in the composition being a CNS disorder-treating amount and the amount of (b) in the composition being sufficient to potentiate the CNS disorder-treating effectiveness of (a).
